(19) United States
(12) Patent Application Publication
(10) Pub. No.: US 2017/0304527 A1
(43) Pub. Date: Oct. 26, 2017

(54) WOUND IRRIGATION SYSTEM

(71) Applicant: Mayo Foundation for Medical Education and Research, Rochester, MN (US)

(72) Inventors: Johnathon M. Aho, Rochester, MN (US); Terry P. Nickerson, Rochester, MN (US); Blake A. Spindler, Rochester, MN (US)

(73) Assignee: Mayo Foundation for Medical Education and Research, Rochester, MN (US)

(21) Appl. No.: 15/516,100

(22) PCT Filed: Oct. 2, 2015
(86) PCT No.: PCT/US15/53650

§ 371 Date: Mar. 31, 2017

Related U.S. Application Data
(60) Provisional application No. 62/059,250, filed on Oct. 3, 2014.

(51) Int. Cl. A61M 3/02; A61M 1/00; A61M 1/28

(52) U.S. Cl. CPC A61M 3/0283; A61M 1/285; A61M 1/0086; A61M 1/0064; A61M 1/0088

(57) ABSTRACT

This document provides devices, systems, and methods for treating patients with wounds. For example, this document provides a multi-lumen tubular device that can deliver wound irrigation, wound suction, and medicinal treatment through the lumens of the tubular device.

### CROSS REFERENCE TO RELATED APPLICATIONS

This application claims the benefit of U.S. Provisional Ser. No. 62/059,250 filed Oct. 3, 2014. This disclosure of the prior application is considered part of (and is incorporated by reference in) the disclosure of this application.

### BACKGROUND

### 1. Technical Field

This document relates to devices, systems, and methods for treating patients with wounds. For example, this document relates to a multi-lumen tubular device that can deliver wound irrigation, wound suction, and medicinal treatment through the lumens of the tubular device.

### 2. Background Information

Unresolved healing of wounds is a significant issue in medicine. Wound irrigation is an essential aspect of wound care. Wound irrigation is meant to remove infectious and/or necrotic debris from tissues, and to assist in wound hydration. Irrigation is considered one of the most effective methods of wound cleansing.

Wound irrigation is the flow of a solution across an open wound to achieve wound hydration, to remove deeper debris, and to assist with the visual examination. The irrigation solution is meant to remove cellular debris and surface pathogens contained in wound exudates or residue from topically applied wound care products. Irrigation is a necessary step in facilitating progression from the inflammatory to the proliferative phase of wound healing, by removing debris that can impede the healing process. When performed properly, wound irrigation can aid in wound healing from the inside tissue layers outward to the skin surface. It may also help prevent premature surface healing over an abscess pocket or infected tract. The goal of irrigation is to clean the wound while avoiding trauma to the wound bed and minimizing the risk of driving bacteria farther into the wound bed.

There is currently no ideal method for irrigating wounds reported in literature. However, current techniques for larger grossly infected wounds include frequent trips to the operating room for assessment of the wound, continued irrigation, and wound care.

### SUMMARY

This document provides devices, systems, and methods for treating patients with wounds. For example, this document provides a multi-lumen tubular device that can deliver wound irrigation, wound suction, and medicinal treatment through the lumens of the tubular device.

In one implementation, a multi-lumen tubular irrigation device for treating a patient with a wound includes an elongate flexible tube and a distal portion coupled to the flexible tube at a distal hub of the distal portion. The flexible tube defines a first lumen that is configured to deliver irrigation fluid to the wound and a second lumen that is configured to provide suction to the wound. The distal portion includes at least one fenestrated irrigate delivery tube extending from the distal hub and in fluid communication with the first lumen, and two or more fenestrated suction tubes extending from the distal hub and in fluid communication with the second lumen.

Such a multi-lumen tubular irrigation device for treating a patient with a wound may optionally include one or more of the following features. The device may further comprise a proximal hub, and the elongate flexible tube may extend from the proximal hub. The proximal hub may include a first connector that is in fluid communication with the first lumen and a second connector that is in fluid communication with the second lumen. The device may further comprise a first auxiliary connector that is in fluid communication with the first lumen and a second auxiliary connector that is in fluid communication with the second lumen. The distal portion may include four or more fenestrated suction tubes extending from the distal hub and in fluid communication with the second lumen.

In another implementation, a medical device system for treating a patient with a wound includes a multi-lumen tubular irrigation device, an irrigation fluid source in fluid communication with the first lumen, and a suction source in fluid communication with the second lumen. The multi-lumen tubular irrigation device comprises an elongate flexible tube and a distal portion coupled to the flexible tube at a distal hub of the distal portion. The flexible tube defines a first lumen that is configured to deliver irrigation fluid to the wound and a second lumen that is configured to provide suction to the wound. The distal portion includes at least one fenestrated irrigate delivery tube extending from the distal hub and in fluid communication with the first lumen, and two or more fenestrated suction tubes extending from the distal hub and in fluid communication with the second lumen.

Such a medical device system for treating a patient with a wound may optionally include one or more of the following features. The irrigation fluid source may be a pump that can operate in pulsatile mode. The device may further comprise a proximal hub, and the elongate flexible tube may extend from the proximal hub. The proximal hub may include a first connector that is configured and operable to be connected with the irrigation fluid source and a second connector that is configured and operable to be connected with the suction source. The system may further comprise a first auxiliary connector that is in fluid communication with the first lumen and a second auxiliary connector that is in fluid communication with the second lumen. The distal portion may include four or more fenestrated suction tubes extending from the distal hub and in fluid communication with the second lumen.

In another implementation, a method for treating a patient with a wound includes: (a) attaching a multi-lumen tubular irrigation device to the patient, (b) sealing a wound covering onto the patient and over the distal portion of the device, (c) delivering irrigation fluid to the wound via the fenestrated irrigate delivery tube, and (d) delivering suction to the wound via the two or more fenestrated suction tubes. The multi-lumen tubular irrigation device includes an elongate flexible tube, and a distal portion coupled to the flexible tube at a distal hub of the distal portion. The flexible tube defines a first lumen that is configured to deliver irrigation fluid to the wound and a second lumen that is configured to provide suction to the wound. The distal portion includes at least one fenestrated irrigate delivery tube extending from the distal hub and in fluid communication with the first lumen, and the distal portion includes two or more fenestrated suction tubes extending from the distal hub and in fluid communication with the second lumen.

Such a method for treating a patient with a wound may optionally include one or more of the following features. The suction delivered to the wound may remove at least some irrigation fluid from the wound. The irrigation fluid may be delivered to the wound using a pump operating in a pulsatile mode. The method may further comprise delivering medications to the wound via the first lumen and via the fenestrated irrigate delivery tube. The device may further comprise a proximal hub, and the elongate flexible tube may extend from the proximal hub. The proximal hub may include a first connector that is connected with a source of the irrigation fluid and a second connector that is connected with a source of the suction. The device may further comprise a first auxiliary connector that is in fluid communication with the first lumen and a second auxiliary connector that is in fluid communication with the second lumen. The distal portion may include four or more fenestrated suction tubes extending from the distal hub and in fluid communication with the second lumen.

In another implementation, a method for performing peritoneal dialysis on a patient includes attaching a multi-lumen tubular irrigation device to the patient, delivering the dialysate solution to the abdomen via the at least one fenestrated dialysate solution delivery tube, allowing the dialysate solution to dwell in the abdomen for a period of time, and delivering suction to the abdomen via the two or more fenestrated dialysate solution suction tubes to remove at least a portion of the dialysate solution from the abdomen. The device comprises an elongate flexible tube, and a distal portion coupled to the flexible tube at a distal hub of the distal portion. The distal portion includes at least one fenestrated dialysate solution delivery tube extending from the distal hub and in fluid communication with the first lumen, and two or more fenestrated dialysate solution suction tubes extending from the distal hub and in fluid communication with the second lumen. The flexible tube defines a first lumen that is configured to deliver dialysate solution to an abdomen of the patient and a second lumen that is configured to provide suction to remove the dialysate solution.

Particular embodiments of the subject matter described in this document can be implemented to realize one or more of the following advantages. In some embodiments, treatment of patients that have wounds can be enhanced using a single device that can provide wound irrigation, wound suction, wound cleansing, and deliver medication to the wound. The use of such a device and treatment technique may reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of patient with an abdominal wound that is undergoing irrigation treatment. The treatment is taking place using a multi-lumen tubular irrigation system in accordance with some embodiments provided herein.

FIG. 2 is a plan view of a multi-lumen tubular irrigation device in accordance with some embodiments provided herein.

FIG. 3 is flowchart of a method for treating a patient with a wound in accordance with some embodiments provided herein.

Like reference numbers represent corresponding parts throughout.

### DETAILED DESCRIPTION

This document provides devices, systems, and methods for treating patients with wounds. For example, this document provides a multi-lumen tubular device that can deliver wound irrigation, wound suction, and medicinal treatment through the lumens of the tubular device. The devices and systems provided herein can be used to treat wounds such as, but not limited to, abdominal, chest, and extremity wounds. In addition, the devices and systems provided herein can be used for peritoneal dialysis access, and in conjunction with negative pressure wound therapy.

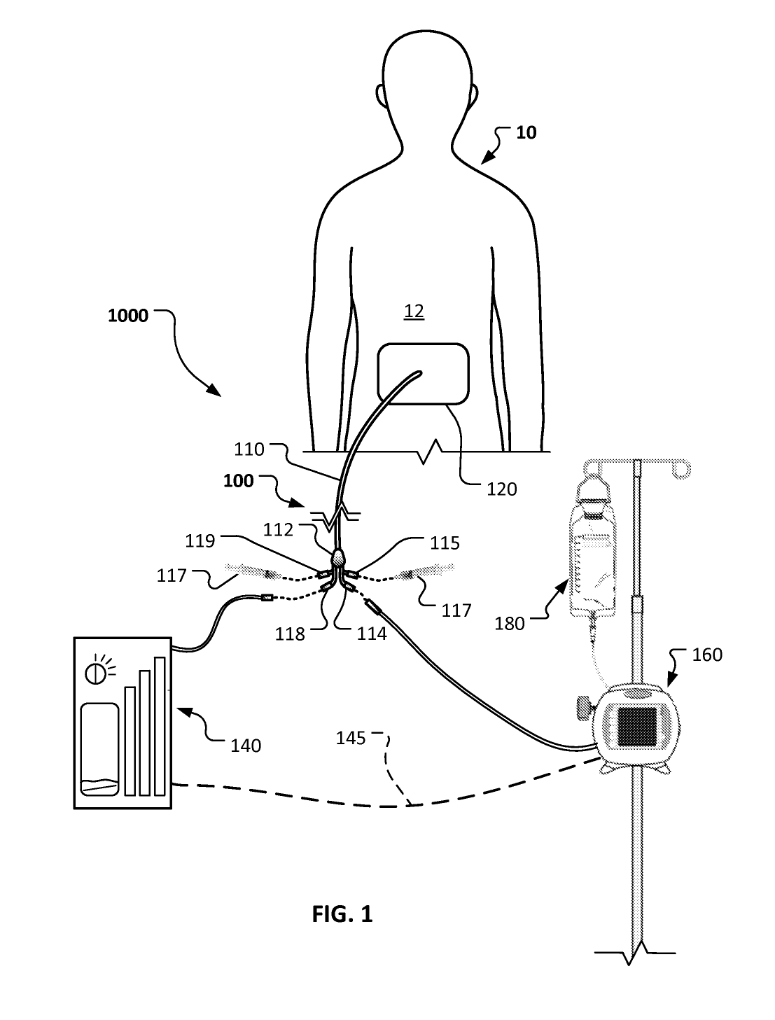

In reference to FIG. 1, a patient 10 with a wound can be treated using an example multi-lumen tubular irrigation device system 1000. The depicted multi-lumen tubular irrigation device system 1000 includes a multi-lumen tubular irrigation device 100, a suction source 140, an irrigation fluid pump system 160, and an irrigation source 180. Multi-lumen tubular irrigation device 100 can optionally be releasably coupled to suction source 140 and irrigation fluid pump system 160 as desired in accordance with a clinician's treatment plan for patient 10.

As will be described further below, in some embodiments multi-lumen tubular irrigation device 100 includes an elongate flexible tube 110 that defines at least two lumens (e.g., one for irrigate solution supply and one for irrigate solution removal by suction), and a proximal hub 112 that separates the lumens. Proximal hub 112 also facilitates the connection of devices to the at least two lumens individually. For example, in the depicted embodiment, the at least two lumens are coupleable using a first lumen connector 114 and a second lumen connector 118 respectively. In the depicted arrangement of multi-lumen tubular irrigation device system 1000, the first lumen connector 114 is used to convey an irrigation fluid to the wound, and the second lumen connector 118 is used to convey a negative air pressure (vacuum) for wound suction and removal of the irrigation fluid from the proximity of the wound. In some implementations, the reverse arrangement can be used. That is, in some implementations the first lumen connector 114 is used to convey a negative air pressure (vacuum) for wound suction and removal of the irrigation fluid from the proximity of the wound, and the second lumen is used to convey an irrigation fluid to the wound connector 118.

In some embodiments, additional connections are included through which medicates (e.g., antibiotics) can be administered to the wound. For example, the depicted embodiment includes a first auxiliary connector 115 that provides access to the first lumen (as does first lumen connector 114), and second auxiliary connector 119 that provides access to the second lumen (as does second lumen connector 118). In some implementations, the first and/or second auxiliary connectors 115 and 119 can be used in conjunction with a syringe 117 (or another device/system) to administer medicates therethrough. In some embodiments, connectors 114, 115, 118, and/or 119 are luer fittings, but any other suitable type of fitting can be used for one or more of connectors 114, 115, 118, and/or 119.

In the example multi-lumen tubular irrigation device system 1000 configuration, a suction source 140 is depicted as a separate bedside type of device. However, it should be understood that suction source 140 can be integrated with, for example, irrigation fluid pump system 160, or may be a connection to a central source of suction such as a hospital wall vacuum source. Irrigation fluid pump system 160 can also be integrated with other devices in some embodiments, such as suction source 140 and/or irrigation source 180. While irrigation source 180 is depicted as a basic solution bag and pole in this example system configuration, irrigation source 180 can be configured differently in some embodiments, including as automated devices that may be integrated with other devices or as a stand-alone device.

In some embodiments, suction source 140 and irrigation fluid pump system 160 are in communication with each other via a data connection 145. Accordingly, in some embodiments of multi-lumen tubular irrigation device system 1000 the operations of suction source 140 and irrigation fluid pump system 160 are coordinated. For example, in some implementations irrigation fluid pump system 160 may run for a period of time prior to the start of suction source 140. In such a case, for example, the wound may be advantageously allowed to soak for the period of time prior to removal of the irrigate solution. Thereafter, suction source 140 may start so as to remove the irrigate and loosened wound contaminants. In another example, suction source 140 may automatically start in response to the start of irrigation fluid pump system 160, and suction source 140 may continue running for a period of time after irrigation fluid pump system 160 has stopped. These are just some examples of how the operations of suction source 140 and irrigation fluid pump system 160 can be coordinated. The operational settings and parameters of suction source 140 and/or irrigation fluid pump system 160 are selectable and programmable by a clinician operator in some embodiments.

In some embodiments, irrigation fluid pump system 160 can operate in pulsatile and/or continuous modes. In some implementations, the pulsatile mode may advantageously facilitate controlled agitation of wound contaminates and serve to enhance removal of such contaminants using multi-lumen tubular irrigation device system 1000.

In some implementations, multi-lumen tubular irrigation device system 1000 is used in conjunction with a seal-tight flexible wound covering 120. At least the periphery of seal-tight flexible wound covering 120 is adhered to the skin 12 of patient 10. Seal-tight flexible wound covering 120 can thereby be used to contain the irrigate solution in the wound area within the periphery of seal-tight flexible wound covering 120, thereby allowing the suction provided by the multi-lumen tubular irrigation device system 1000 to remove much of the irrigate solution from the wound. In some implementations, seal-tight flexible wound covering 120 is applied to the skin 12 of patient 10 while multi-lumen tubular irrigation device system 1000 is being used to perform the wound irrigation process, and seal-tight flexible wound covering 120 is removed thereafter. In some implementations, seal-tight flexible wound covering 120 remains on the skin 12 of patient 10 after performance of wound irrigation using multi-lumen tubular irrigation device system 1000, and can be used multiple times in conjunction with multi-lumen tubular irrigation device system 1000.

In some implementations, seal-tight flexible wound covering 120 includes an opening through which elongate flexible tube 110 is passed. In some implementations, elongate flexible tube 110 is passed into the wound area via the peripheral seal of seal-tight flexible wound covering 120. That is, multi-lumen tubular irrigation device 100 is placed in the wound area and seal-tight flexible wound covering 120 is adhered to the skin 12 of patient 10 so as to fully encompass multi-lumen tubular irrigation device 100 except for a segment of elongate flexible tube 110 that extends away from the wound area under the peripheral seal of seal-tight flexible wound covering 120.

The periphery of seal-tight flexible covering 120 surrounds the wound being treated. In the depicted embodiment, the periphery of seal-tight flexible covering 120 is generally rectangular. However, other shapes can be used for the periphery of seal-tight flexible covering 120, such as, but not limited to, square, circular, ovular, polygonal, and the like. In some implementations, the periphery of seal-tight flexible covering 120 may be cut by a clinician to suit the particular topography at the site of installation on patient 10.

The periphery of seal-tight flexible covering 120 is sealed to skin 12 of patient 10. In some implementations, a medical adhesive is used to seal seal-tight flexible covering 120 to skin 12. For example, in particular implementations a TEGADERM® film adhesive can be used to seal seal-tight flexible covering 120 to skin 12. Using such a seal, the wound is segregated by seal-tight flexible covering 120 from other portions of skin 12 to protect the other portions of skin 12 from contamination and irritation from the irrigate solution and associated contaminants. In addition, the seal allows for effective wound irrigation and enhanced suction by multi-lumen tubular irrigation device 100.

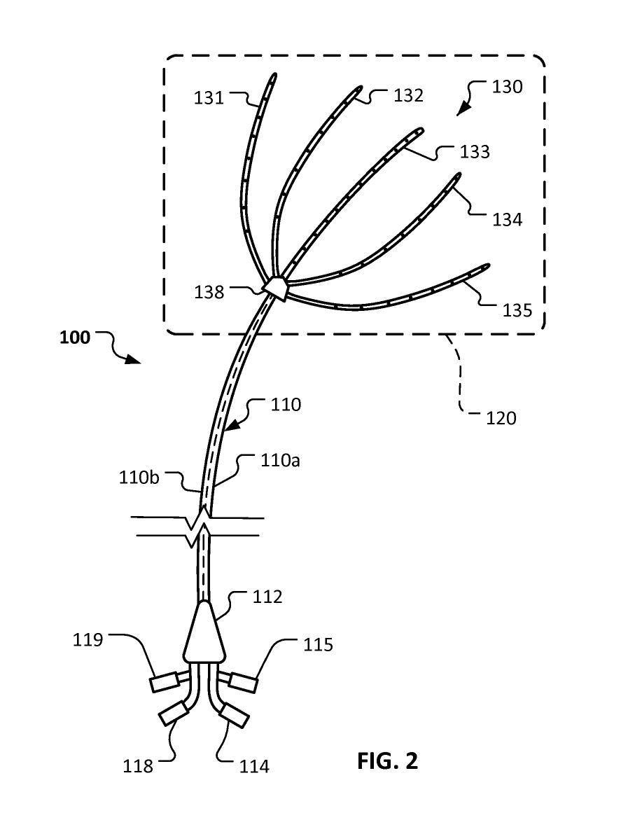

In FIG. 2, multi-lumen tubular irrigation device 100 is shown in greater detail. Multi-lumen tubular irrigation device 100 includes elongate flexible tube 110, proximal hub 112, and a distal portion 130. Distal portion 130 is depicted as within the periphery of seal-tight flexible covering 120. As described above, elongate flexible tube 110 includes at least two lumens, which are identified here as a first lumen 110a and a second lumen 110b. In the depicted embodiment of multi-lumen tubular irrigation device 100, first lumen 110a is in fluid communication with connectors 114 and 115, and second lumen 110b is in fluid communication with connectors 118 and 119.

In the depicted embodiment, distal portion 130 includes a distal hub 138, a first fenestrated tube 131, a second fenestrated tube 132, a third fenestrated tube 133, a fourth fenestrated tube 134, and a fifth fenestrated tube 135. While the depicted embodiment of multi-lumen tubular irrigation device 100 includes five fenestrated tubes 131, 132, 133, 134, and 135, in some embodiments fewer or more than five fenestrated tubes are included. For example, in some embodiments two, three, four, six, seven, eight, nine, ten, or more than ten fenestrated tubes are included.

In some implementations, some of the fenestrated tubes 131, 132, 133, 134, or 135 may be larger in diameter than others of the fenestrated tubes 131, 132, 133, 134, or 135. In some implementations, some of the fenestrated tubes 131, 132, 133, 134, or 135 may be longer than others of the fenestrated tubes 131, 132, 133, 134, or 135. In some implementations, the fenestrated tubes 131, 132, 133, 134, or 135 can be trimmed in length to suit the particular implementation as desired by a clinician.

The fenestrated tubes 131, 132, 133, 134, and 135 each include one or more fenestrations. The fenestrations provide outlets through which irrigate solution can flow to the wound area, or inlets for suctioning the irrigate solution from the wound area. Such fenestrations can be circular holes, ovular holes, slots, and the like, and combinations thereof. Some fenestrated tubes 131, 132, 133, 134, or 135 may have a different pattern or sizes of fenestrations than other fenestrated tubes 131, 132, 133, 134, or 135.

In the depicted embodiment, fenestrated tubes 131, 132, 133, 134, and 135 each extend distally from distal hub 138. Distal hub 138 is coupled to elongate flexible tube 110, and connects fenestrated tubes 131, 132, 133, 134, and 135 with first lumen 110a or second lumen 110b. For example, in some embodiments first lumen 110a is in fluid communication (via distal hub 138) with third fenestrated tube 133, while second lumen 110b is in fluid communication (via distal hub 138) with first fenestrated tube 131, second fenestrated tube 132, fourth fenestrated tube 134, and fifth fenestrated tube 135. In another example, in some embodiments first lumen 110a is in fluid communication (via distal hub 138) with second fenestrated tube 132 and fourth fenestrated tube 134, while second lumen 110b is in fluid communication (via distal hub 138) with first fenestrated tube 131, third fenestrated tube 133, and fifth fenestrated tube 135. All other possible arrangements of connections between the lumens of elongate flexible tube 110 and the fenestrated tubes of distal portion 130 are also within the scope of this disclosure.

In one example implementation, third fenestrated tube 133 is in fluid communication with first lumen 110a. Connector 114 allows connection of a pressurized source of irrigate solution (e.g., irrigation fluid pump system 160 of FIG. 1) to first lumen 110a and thereby to third fenestrated tube 133. Second lumen 110b is in fluid communication with first fenestrated tube 131, second fenestrated tube 132, fourth fenestrated tube 134, and fifth fenestrated tube 135. Connector 118 allows connection of a suction source (e.g., suction source 140 of FIG. 1) to second lumen 110b and thereby to first fenestrated tube 131, second fenestrated tube 132, fourth fenestrated tube 134, and fifth fenestrated tube 135. Using this example arrangement of multi-lumen tubular irrigation device 100, irrigate solution can be expelled from the fenestrations of third fenestrated tube 133 and into the wound area. The irritate solution can be expelled using a pulsatile mode, continuously, or a combination thereof Thereafter, the irrigate solution and loosened debris can be suctioned away from the wound area via the fenestrations of first fenestrated tube 131, second fenestrated tube 132, fourth fenestrated tube 134, and fifth fenestrated tube 135. The level of suction (negative pressure or vacuum level) can be adjusted as desired by the clinician operator.

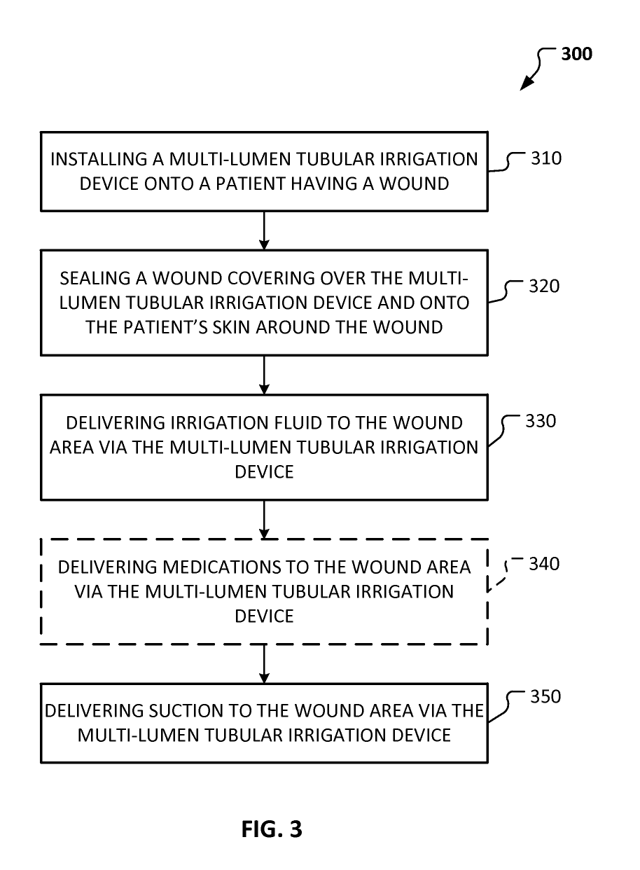

In reference to FIG. 3, a method 300 is provided for treating a patient with a wound, in accordance with some embodiments provided herein. Method 300 can include the use of multi-lumen tubular irrigation device 100, and/or multi-lumen tubular irrigation device system 1000 as described above. In some implementations of method 300, multi-lumen tubular irrigation device 100 can be used in combination with another type of suction source (e.g., wall suction) and/or another type of irrigation fluid source.

At operation 310, a multi-lumen tubular irrigation device is installed on the patient in the area of a wound. For example, a distal portion of the multi-lumen tubular irrigation device having multiple fenestrated tubes can be placed in the wound area as depicted in FIG. 2.

At operation 320, a wound covering is installed on the patient over the distal portion of the multi-lumen tubular irrigation device. In some implementations, the periphery of the wound covering is sealed to the patient using adhesives such that the periphery of the wound covering circumscribes the wound (and the distal portion of the multi-lumen tubular irrigation device) to create a substantially liquid-tight seal. The wound covering can face the skin of the patient to create a space between the wound covering and the skin/wound of the patient. The distal portion of the multi-lumen tubular irrigation device can reside within the space.

At operation 330, an irrigation fluid is delivered to the wound area of the patient through a lumen of the multi-lumen tubular irrigation device. The irrigation fluid can be delivered via fenestrations of one or more tubes located at a distal portion of the multi-lumen tubular irrigation device.

At operation 340, medications (e.g., antibiotics) can be optionally delivered to the wound area of the patient through a lumen of the multi-lumen tubular irrigation device. In some implementations of method 300, the medications can be delivered from a syringe, or from another source of medication and into the same lumen that carries the irrigation fluid. In particular implementations, the irrigation fluid can be delivered via another lumen of the multi-lumen tubular irrigation device.

At operation 350, suction to remove irrigation fluids, debris, and/or contaminants from the wound area can be delivered to the patient through a lumen of the multi-lumen tubular irrigation device. In some implementations of method 300, the suction can be delivered to the wound area via fenestrations of one or more tubes located at a distal portion of the multi-lumen tubular irrigation device.

In another type of implementation, peritoneal dialysis can be performed using the multi-lumen tubular irrigation devices provided herein. During the performance of peritoneal dialysis, a sterile cleansing solution (dialysate) flows through the catheter (e.g., one or more of the fenestrated tubes) into a patient's abdomen. The solution stays in the patient's abdomen for a prescribed period of time, known as dwell time. During this dwell time, waste, chemicals and extra fluid in the patient's blood pass from tiny blood vessels (capillaries) in the lining of the abdominal cavity (peritoneum) into the dialysis solution. In some implementations, the solution contains a sugar that draws wastes and extra fluid through the capillaries in the patient's peritoneum into the patient's abdomen.

When the dwell time is over, the solution, along with waste products drawn from the patient's blood and any excess fluid, drains into a sterile collection bag via other fenestrated tubes of the multi-lumen tubular irrigation devices provided herein. The process of filling and then draining the abdomen is called an exchange.

Different methods of peritoneal dialysis have different schedules of daily exchanges. The two main schedules are continuous ambulatory peritoneal dialysis (CAPD) and continuous cycling peritoneal dialysis (CCPD). In some implementations, a combination of both methods are used.

With CCPD, which may also be called automated peritoneal dialysis (APD), a machine called an automated cycler performs three to five exchanges at night while the patient sleeps. The cycler automatically fills the patient's abdomen with dialysis solution, allows it to dwell there and then drains it to a sterile drainage bag that needs to be emptied in the morning. In some cases, the patient must remain attached to the machine for 10 to 12 hours at night. In the morning, the patient begins one exchange with a dwell time that lasts the entire day. The patient is not connected to the machine during the day. From the description provided above, one or ordinary skill in the art can envision how the multi-lumen tubular irrigation devices provided herein can be used to perform peritoneal dialysis techniques.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

1. A multi-lumen tubular irrigation device for treating a patient with a wound, the device comprising:
an elongate flexible tube, the flexible tube defining a first lumen that is configured to deliver irrigation fluid to the wound and a second lumen that is configured to provide suction to the wound; and
a distal portion coupled to the flexible tube at a distal hub of the distal portion, the distal portion including at least one fenestrated irrigate delivery tube extending from the distal hub and in fluid communication with the first lumen, and the distal portion including two or more fenestrated suction tubes extending from the distal hub and in fluid communication with the second lumen.

2. The device of claim 1, further comprising a proximal hub, and wherein the elongate flexible tube extends from the proximal hub.

3. The device of claim 2, wherein the proximal hub includes a first connector that is in fluid communication with the first lumen and a second connector that is in fluid communication with the second lumen.

4. The device of claim 3, further comprising a first auxiliary connector that is in fluid communication with the first lumen and a second auxiliary connector that is in fluid communication with the second lumen.

5. The device of claim 1, wherein the distal portion includes four or more fenestrated suction tubes extending from the distal hub and in fluid communication with the second lumen.

6. A medical device system for treating a patient with a wound, the system comprising:
a multi-lumen tubular irrigation device, the device comprising:
an elongate flexible tube, the flexible tube defining a first lumen that is configured to deliver irrigation fluid to the wound and a second lumen that is configured to provide suction to the wound; and
a distal portion coupled to the flexible tube at a distal hub of the distal portion, the distal portion including at least one fenestrated irrigate delivery tube extending from the distal hub and in fluid communication with the first lumen, and the distal portion including two or more fenestrated suction tubes extending from the distal hub and in fluid communication with the second lumen;
an irrigation fluid source in fluid communication with the first lumen; and
a suction source in fluid communication with the second lumen.

7. The system of claim 6, wherein the irrigation fluid source is a pump that can operate in pulsatile mode.

8. The system of claim 6, wherein the device further comprises a proximal hub, and wherein the elongate flexible tube extends from the proximal hub.

9. The system of claim 8, wherein the proximal hub includes a first connector that is configured and operable to be connected with the irrigation fluid source and a second connector that is configured and operable to be connected with the suction source.

10. The system of claim 8, further comprising a first auxiliary connector that is in fluid communication with the first lumen and a second auxiliary connector that is in fluid communication with the second lumen.

11. The system of claim 6, wherein the distal portion includes four or more fenestrated suction tubes extending from the distal hub and in fluid communication with the second lumen.

12. A method for treating a patient with a wound, the method comprising:
attaching a multi-lumen tubular irrigation device to the patient, the device comprising:
an elongate flexible tube, the flexible tube defining a first lumen that is configured to deliver irrigation fluid to the wound and a second lumen that is configured to provide suction to the wound; and
a distal portion coupled to the flexible tube at a distal hub of the distal portion, the distal portion including at least one fenestrated irrigate delivery tube extending from the distal hub and in fluid communication with the first lumen, and the distal portion including two or more fenestrated suction tubes extending from the distal hub and in fluid communication with the second lumen;
sealing a wound covering onto the patient and over the distal portion of the device;
delivering irrigation fluid to the wound via the fenestrated irrigate delivery tube; and
delivering suction to the wound via the two or more fenestrated suction tubes.

13. The method of claim 12, wherein the suction delivered to the wound removes at least some irrigation fluid from the wound.

14. The method of claim 12, wherein the irrigation fluid is delivered to the wound using a pump operating in a pulsatile mode.

15. The method of claim 12, further comprising delivering medications to the wound via the first lumen and via the fenestrated irrigate delivery tube.

16. The method of claim 12, wherein the device further comprises a proximal hub, and wherein the elongate flexible tube extends from the proximal hub.

17. The method of claim 16, wherein the proximal hub includes a first connector that is connected with a source of the irrigation fluid and a second connector that is connected with a source of the suction.

18. The method of claim 12, wherein the device further comprises a first auxiliary connector that is in fluid communication with the first lumen and a second auxiliary connector that is in fluid communication with the second lumen.

19. The method of claim 12, wherein the distal portion includes four or more fenestrated suction tubes extending from the distal hub and in fluid communication with the second lumen.

20. (canceled)
